(19) United States
(12) Patent Application Publication
(10) Pub. No.: US 2010/0028384 A1
(43) Pub. Date: Feb. 4, 2010

(54) YEAST EXPRESSED CLASSICAL SWINE FEVER VIRUS GLYCOPROTEIN E2 AND USE THEREOF

(75) Inventors: Chienjin Huang, Taichung (TW); Maw-Sheng Chien, Taichung (TW); Guang-Jan Lin, Taichung (TW)

Correspondence Address: PAI PATENT & TRADEMARK LAW FIRM, 1001 FOURTH AVENUE, SUITE 3200, SEATTLE, WA, 98154, US

(73) Assignee: MAO-XING BIOLOGICAL TECHNOLOGY CO., LTD., Taipei Hsien (TW)

(21) Appl. No.: 12/512,049

(22) Filed: Jul. 30, 2009

(30) Foreign Application Priority Data
Jul. 31, 2008 (TW) 097129117

(51) Int. Cl. A61K 39/12; C12P 21/00

(52) U.S. Cl. 424/218.1; 435/69.1

(57) ABSTRACT

A glycoprotein E2 of classical swine fever virus (CSFV) expressed in a recombinant yeast system. The recombinant E2 protein (yE2) is able to form a homodimer, exhibits glycosylation conformation and possesses correct immunogenicity. An anti-CSFV vaccine can be provided with yE2 as a major active ingredient to induce high titers of neutralizing antibody in vaccinated pigs, and to induce a protection against CSFV infection.

### FIELD OF THE INVENTION

The present invention relates to provision of a recombinant yeast system for expressing the glycoprotein E2 of classical swine fever virus (CSFV). The expressed recombinant E2 protein (yE2) is characterized by the ability to form a homodimer and exhibits glycosylation conformation and possesses correct immunogenicity. The present invention further provides an anti-CSFV vaccine comprising yE2 as a major active ingredient, which can induce high titers of neutralizing antibody in vaccinated pigs and is able to induce a protection against CSFV infection.

### BACKGROUND OF THE INVENTION

Classical swine fever virus (CSFV) is a virus of the genus Pestivirus in the family Flaviviridae (Leyssen et al., 2000, Clin. Microbiol. Rev. 13, 67-82). The infection by CSFV in pigs causes clinical symptoms such as fever and bleeding. It is highly infectious and lethal, which can cause economic damage to animal husbandry (Vilcek et al., 1996, Virus Res. 43, 137-147). The genome of CSFV consists of a (+) RNA of 12.5 kb encoding a giant polyprotein, which is digested into mature viral structural and non-structural proteins by protease of the host cell or the virus (Chamber et al., 1990, Annu. Rev. Microbiol. 44, 649-688). The structural proteins of CSFV include nucleocapsid protein C, envelope glycoproteins E^{rns}, E1, and E2 (Dong & Chen, 2007, Vaccine 25, 205-230). Among these, E2 and E^{rns} have been proved to have the ability to induce neutralizing antibody production in a host (see, for example, Bouma et al., 2000, Vaccine 18, 1374-1381; Konig et al., 1995, J. Virol. 69, 6479-6486; van Rijn et al., 1993, J. Gen. Virol. 74, 2053-2060; and Weiland et al., 1992, J. Virol. 66, 3677-3682).

CSFV glycoprotein E2 is the major viral antigen for inducing neutralizing antibody production in pigs. Therefore, E2 is the target protein in the development of CSFV vaccines. Recently, E2 subunit vaccine has been successfully produced by an insect cell expression system infected with baculovirus (Hulst et al., 1994, Virology 2000, 558-565; Bouma et al., 2000, supra; and van Oers et al., 2001, J. Biotechnol. 86, 31-38). E2 subunit vaccine can not only protect pigs against CSFV infection, but may be used to distinguish the immunized pigs from CSFV-infected pigs by detecting anti-E^{rns} and E2 antibodies (de Smit et al., 2000, Vet. Q. 22, 182-188; Floegel-Niesmann, 2001, Vet. Microbiol. 83, 121-136; and Moormann et al., 2000, Vet. Microbiol. 73, 209-219). It is the most important advantage of the marker vaccine. However, the procedure of insect cell expression is very complex, laborious, easy to be contaminated and costly, which is the major problem in large scale production.

Previously, the inventor has successfully produced active E^{rns} protein by a yeast Pichia pastoris expression system (Huang et al., 2006, J. Virol. Methods 132, 40-47). The yeast expression system possesses the characteristics of cultivating at high density and in a cheaper medium, and, especially, can perform the glycosylation modification as in eukaryotes to largely produce the desired glycoprotein at high efficiency and low cost. Accordingly, the aim of the present invention is to prepare recombinant glycoprotein E2 of classical swine fever virus by using a yeast (for example, Pichia pastoris) expression system. It is also another aim of the present invention to evaluate the efficacy of the present vaccine in vaccination and viral challenge tests for further development of efficacious CSF subunit marker vaccine with advantages of easy manipulation and low cost.

### SUMMARY OF THE INVENTION

One of the objects of the invention is to provide a process for producing glycoprotein E2 of classical swine fever virus (yE2) by using a recombinant yeast expression system. The process comprises: cloning the gene fragment of glycoprotein E2 of CSFV into the yeast expression vector pGAPZαC (Invitrogen) to construct a recombinant expression plasmid; transforming the obtained recombinant expression plasmid into Pichia pastoris host cells; cultivating the transformant cells under an appropriate condition for the expression and secretion of the yE2 glycoprotein into a culture medium; and isolating and purifying the recombinant yE2 glycoprotein from the supernatant of the culture medium. In one embodiment, the recombinant expression plasmid is pGAPZαC/E2.

Another object of the invention is to provide a recombinant glycoprotein E2 of classical swine fever virus (yE2) produced in a yeast expression system. The recombinant E2 protein (yE2) is characterized by the ability to form a homodimer and to exhibit glycosylation conformation and correct immunogenicity.

Yet another object of the invention is to provide a subunit vaccine for protecting pigs from the infection by CSFV, which comprises a recombinant glycoprotein E2 of classical swine fever virus produced in yeast expression system (namely, yE2), and a veterinary acceptable adjuvant. In an embodiment of the invention, the recombinant yE2 subunit vaccine can induce production of high titer neutralizing antibody, and is able to induce a protection against CSFV infection.

The other features of the invention will become apparent in the course of the detailed disclosure of the following embodiments.

### BRIEF DESCRIPTION OF DRAWINGS

FIG. 1 shows the the construction of the yeast recombinant expression plasmid pGAPZαC/E2. The CSFV E2 gene fragment encoding the amino acid residues 1-342 was cloned into the yeast Pichia pastoris expression vector pGAPZαC via ClaI/XbaI cloning sites. The E2 was expressed as fusion to an N-terminal peptide coding the Saccharomyces cerevisiae α-factor secretion signal, and a C-terminal peptide containing the myc epitope and a polyhistidine tag.

FIG. 2 shows the Western blot analysis result of the yeast expressed recombinant glycoprotein E2 of classical swine fever virus (yE2). Wild-type yeast secretory protein (WT) and yE2 are subjected to electrophoresis under the condition with (+) or without (−) 5% β-mercaptoethanol, or are treated with enzyme PNGase for de-glycosylation, and then analyzed by Western blotting with specific anti-E2 monoclonal antibody WH303. The arrows indicate homodimer and monomer of yE2 protein, respectively. The deglycosylated yE2 is indicated by an asterisk.

FIG. 3 shows the neutralizing titer in blood samples taken in vaccination and viral challenge periods. The animals were booster vaccinated at 3 weeks after the first vaccination, and then subjected to challenge infection test at 10 weeks after the booster vaccination. Blood samples were taken at intervals of two weeks after booster vaccination, and once every two days during the viral challenge. The neutralizing titer is the highest dilution ratio that antiserum of pigs can completely neutralize 200 TCID₅₀ CSFV, and is presented as a value of log₂. Nos. 1˜4 are yE2-vaccinated, and Nos. 5˜6 are control.

FIG. 4 shows the changes in body temperature during the challenge infection test. The rectal body temperature of animals in yE2-vaccinated group (No. 1˜4) and control group (No. 5˜6) were measured and recorded every day before and during the course of the challenge infection test. The changes in body temperature were recorded until day 14 after the viral challenge, or discontinued for those control pigs that were euthanized due to severe clinical symptoms.

FIG. 5 shows the changes in WBC (white blood cell) count in blood samples after challenge infection by CSFV. EDTA blood was taken from tested pigs once every two days from day 1 to day 16 after challenge infection. The WBC count in the blood samples is calculated in a semi-automatic blood cell counter (Sysmex F-800).

FIG. 6 shows the production of anti-E^{rns} and anti-E2 antibodies in pigs after vaccination and viral challenge infection. Six SPF pigs were randomly divided into yE2-vaccinated group and control group, and numbered. No. 1 through No. 4 were immunized with yE2. No. 5 and No. 6, as control, were immunized with the supernatant from non-recombinant yeast culture. The animals were booster vaccinated at 3 weeks after the first vaccination, and then subjected to viral challenge infection test at 10 weeks after the booster vaccination. Blood samples were taken at intervals of two weeks after booster vaccination, and once every two days during the viral challenge test. The titer of anti-E2 (A) and E^{rns} (B) antibody in serum samples were analyzed by using blocking ELISA (CSFV antibody testing kit, Idexx Laboratory) and CSFV marker ELISA (CHEKIT CSFV marker ELISA, Idexx Laboratory), respectively.

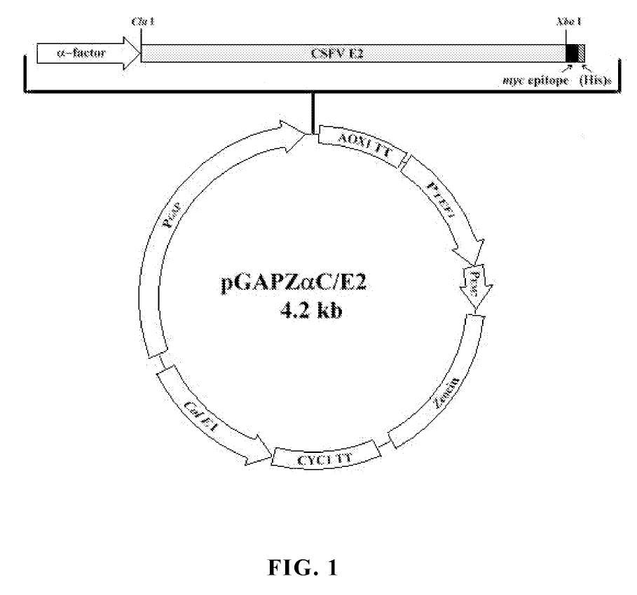

### DETAILED DESCRIPTION OF THE INVENTION

The preferred embodiments of the invention will be described as follows. These examples are given for illustration of the invention and are not intended to be limiting. It will be apparent to those skilled in the art that many modifications may be practiced without departing from the purpose and interest of this invention.

### EXAMPLE 1

### Preparation of Recombinant Glycoprotein E2 of Classical Swine Fever Virus in Yeast Secreting Expression System

A. Construction of Recombinant E2 Expression Vector

The recombinant plasmid pENTR-E2 containing E2 gene fragment of CSFV vaccine strain LPC had been constructed previously in our laboratory. In this experiment, an E2 gene fragment with ClaI and XbaI resrtiction sites was amplified by polymerase chain reaction (PCR) using the plasmid pENTR-E2 as template and a pair of E2 gene specific primers yE2fl: TTATCGATTCGGCTAGCCTGCAAG (SEQ ID NO:3), and yE2dCr: CGCTCTAGAAATTCTGCGAAGTA (SEQ ID NO:4), in a pre-heated thermocycler (GeneAmp PCR system 9700; Perkin-Elmer). Each primer contains restriction enzyme recognition sequences of ClaI or XbaI which are underlined. Conditions of the PCR reaction were set up as follow: 94° C. for 5 min; 30 cycles consisting of 94° C. for 40 sec, 53° C. for 40 sec, and 72° C. for 1 minute. Final extension is carried out at 72° C. for 7 minutes. The PCR is conducted in a reaction mixture of 50 μl total volume, which consisted of 75 mM Tris-HCl (pH 8.8), 20 mM (NH₄)₂SO₄, 0.01% Tween 20, 1.5 mM MgCl₂, 200 μM of dNTP's mixture (dATP, dTTP, dCTP, dGTP), 1.25 units of Pfu DNA polymerase (MBI Fermentas), 10 pmol of primers, 1 ng of pENTR-E2 plasmid DNA. The Pfu DNA polymerase with proofreading activity was selected for use in PCR in order to amplify absolutely correct E2 gene sequence. The amplified PCR product was isolated by agarose gel electrophoresis, and the E2 gene fragment was recovered from the gel. The DNA fragment was digested with restriction enzymes ClaI and XbaI, then cloned into yeast expression vector pGAPZαC (Invitrogen) to obtain pGAPZαC/E2 (deposited with the Agricultural Research Culture Collection, on Jul. 28, 2008, as Deposit No. NRRL Y-50161), and the sequence thereof (SEQ ID NO: 1) was confirmed to be right by DNA sequencing.

B. Transformation and Selection of Yeast Transformants

According to the method described by Becker and Guarente in Methods Enzymol. 194, 182-187, 1991, the constructed plasmid DNA was transformed into Pichia pastoris SMD1168 (Invitrogen) by electroporation. 30 μg of linear pGAPZαC/E2 plasmid DNA, which had been digested with BspHI, and SMD1168 cells were placed in a 0.2 ml sterile cuvette, and subjected to electroporation under the conditions of 1.7 kV, 25 μF, and 200 ohms by Gene pulser (Bio-Rad). Subsequently, the SMD1168 cells were plated onto yeast extract peptone dextrose (YPD; 1% yeast extract, 2% peptone, 2% dextrose) medium agar plate containing 100 μg/ml of Zeocin (Invitrogen), and cultivated at 30° C. for 2 to 3 days until colony formation. A single colony was picked and the sequence accuracy of inserted E2 gene fragment in the yeast chromosomal DNA was examined by PCR (Li et al, 2001, Protein Express. Puri. 24, 438-445).

C. Expression of Recombinant E2 Protein

The recombinant yeast was cultivated in 1 ml YPD liquid medium at 30° C., 250 rpm for 16 hr. 0.1 ml of the culture was transferred into a baffled flask with 50 ml of fresh YPD liquid medium. After further cultivation for 4 days, the supernatant was collected by centrifugation at 12,000×g, at 4° C. for 20 min. Recombinant E2 protein was purified from the collected supernatant by 60% ammonia sulfate precipitation, and the protein concentration thereof was determined by Bradford protein analysis kit (Bio-Rad), then stored at −80° C.

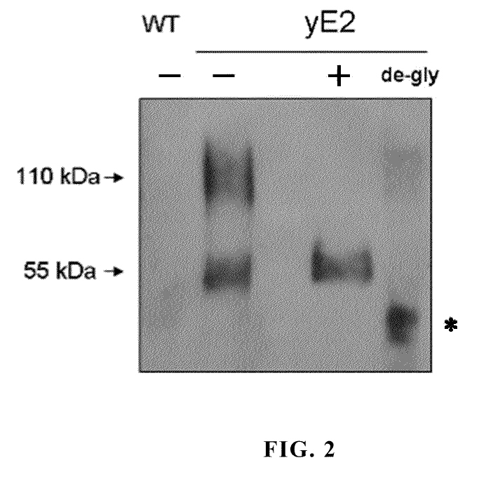

Recombinant glycoprotein E2 of classical swine fever virus (yE2) was successfully expressed by the yeast secreting expression system and secreted into the culture supernatant. Because the recombinant yE2 protein does not have the transmembrane region of 31 amino acid residues at the C-terminus of the E2 protein, it is beneficial to the protein expression and purification in further steps. The results of Western blot analysis using a monoclonal antibody WH303 specific to CSFV E2 showed that monoclonal antibody WH303 could recognize two secretory proteins (with molecular weight of 110 kDa and 55 kDa, respectively) in the supernatant. Only the protein with molecular weight of 55 kDa could be recognized after adding the reducing agent β-mercaptoethanol (as shown in FIG. 2). It suggests that the molecular weight of yE2 protein is about 55 kDa, and it may form a homodimer of about 110 kDa. SEQ ID NO: 2 shows the predicted amino acid sequence of yE2. The yE2 protein has the same amino acid residues 1-342 as the E2 of CSFV vaccine strain LPC, and additionally contains the sequence of an expression vector (corresponding to amino acid residues 343-364 of SEQ ID NO:2) at the C-terminus, which encodes a myc epitope (amino acid residues 344-352) and a 6× Histidine tag (amino acid residues 359-364) for further identification and purification of expressed recombinant protein.

In addition, deglycosylation using the peptide N-glycosylase F (PNGase; New England Biolabs) to remove the N-glycosylation on recombinant yE2 glycoprotein was also performed. Briefly, the mixture of culture supernatant and denaturing buffer containing 0.5% SDS and 1% β-mercaptoethanol was incubated at 100° C. for 10 min. 1 μl of PNGase in reaction buffer (0.05 M sodium phosphate [pH7.5], 1% NP-40) was added to the mixture after the mixture had been cooled to room temperature, and incubated at 37° C. for 30 min for further analysis by Western blotting.

As the results show in FIG. 2, yE2 moved more quickly after deglycosylation by PNGase, which indicates that it resulted in a smaller molecular weight. The results also prove that the yE2 produced by Pichia pastoris yeast expression system actually underwent N-glycosylation modification.

### EXAMPLE 2

### yE2 Immunization and Viral Challenge Test in Pigs

Six-week-old specified pathogen-free (SPF) piglets were immunized with yE2 protein or control antigen, and boosted once after three weeks. The production of antibody in immunized pigs was detected by neutralization reaction and ELISA. Six SPF piglets (6-week-old) were randomly divided into yE2-vaccinated group (n=4) and control group (n=2). 1 mg of concentrate from the supernatant of wild type yeast culture or pGAPZαC/E2 transformed recombinant yeast culture was mixed homogeneously with equal volume of IMS 1113 (SEPPIC) adjuvant, and then injected intramuscularly into the neck to vaccinate each pig in both of the two groups when the animals were six and nine weeks old, respectively. Serum blood samples were collected from each pig before vaccination and every two weeks after immunization for analyzing antibody production.

Neutralizing titer was determined by the neutralizing test in a micro-plate system, and the virus was detected with indirect immuno-fluorescence staining. The serum was subjected to complement inactivation (56° C., 30 min) before performing 2-fold dilutions. 50 μl of the diluted serum was co-incubated with equal volume of viral solution of 200 TCID₅₀ CSFV LPC isolate at 37° C. for 1 hr. 1×10⁴ PK-15 cells in 100 μl of growth medium was added to each well, and cultured at 37° C. for 72 hr. Cells were fixed with 10% formalin (FISH), and incubated with monoclonal antibody WH303. After washing, the cells were incubated with 1000× dilution of Alexa Flour 488 conjugated goat anti-mouse IgG antibody (molecular probes), and observed under an inverted fluorescent microscope. The neutralizing titer is determined as the highest dilution rate that shows no fluorescence signal, and presented as log₂ value.

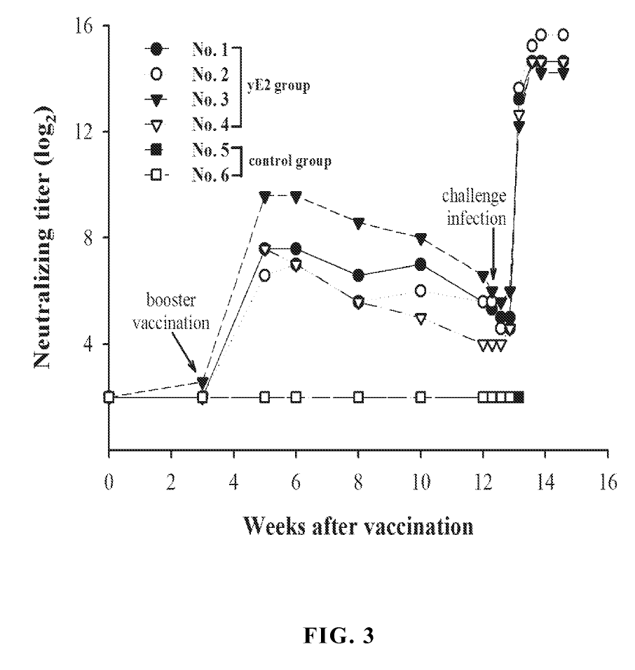

No adverse side effect was observed in immunized pigs, which indicates the high safety of the subunit vaccine. Furthermore, as showed in FIG. 3, all yE2-vaccinated pigs could mount an anamnestic response at 2 weeks after booster vaccination with the neutralizing antibody titers ranging from 2⁷ to 2¹⁰, which is significantly higher than the protective titer of 2⁵. The titers remained above 2⁵ in three of four pigs for 8 weeks, whereas in pig no. 4, the titer declined to 2⁴ at the time of challenge infection. Neutralizing antibody titers at one week post challenge infection ranged from 2¹² to 2¹⁴ and even to 2¹⁶ one week later in all yE2-vaccinated pigs. In general, as the titer of neutralizing antibody is equal to or higher than 2⁵, it is protective to CSFV in pigs. The yE2 subunit vaccine could effectively elicit continuous production of protective neutralizing antibody, and elicit a large amount of neutralizing antibody in a short time after the challenge infection with a high dose of virulent CSFV isolate.

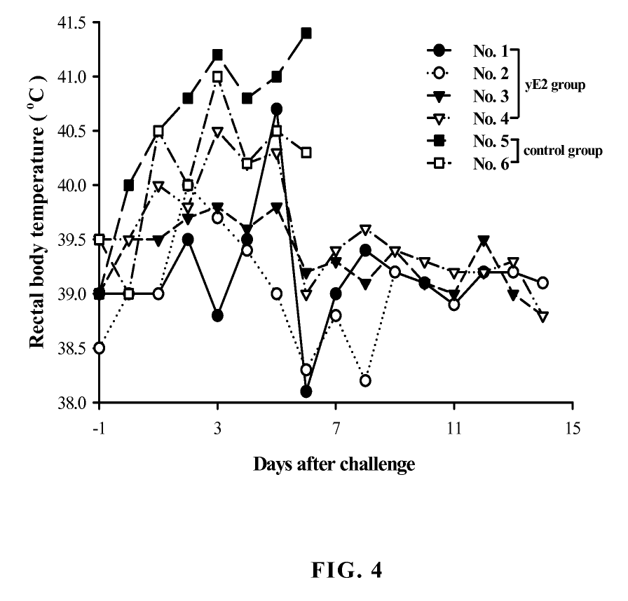

Each pig was subjected to challenge infection by injecting intramuscularly at the neck with 2 ml of 1×10⁵ TCID₅₀ virulent CSFV isolate at 10 weeks after the booster vaccination. Clinical symptoms and changes in rectal body temperature were observed and recorded every day. In addition, EDTA whole blood and serum were taken from tested pigs once every two days for use in WBC counting and serological analysis. Pigs were euthanized at 2 weeks after challenge infection, and anatomized for further examination. As shown in FIG. 4, the control pigs developed an acute febrile response (40.5-41.4° C.) lasting for 6 days after challenge infection, and had to be euthanized due to severe clinical symptoms at day 6. In contrast, with the exception of pig no. 4 which demonstrated a mild febrile response (40.2-40.5° C.) lasting from post-infection day 3 to post-infection day 5, none of the yE2-vaccinated pigs became febrile.

### EXAMPLE 3

### WBC Counting

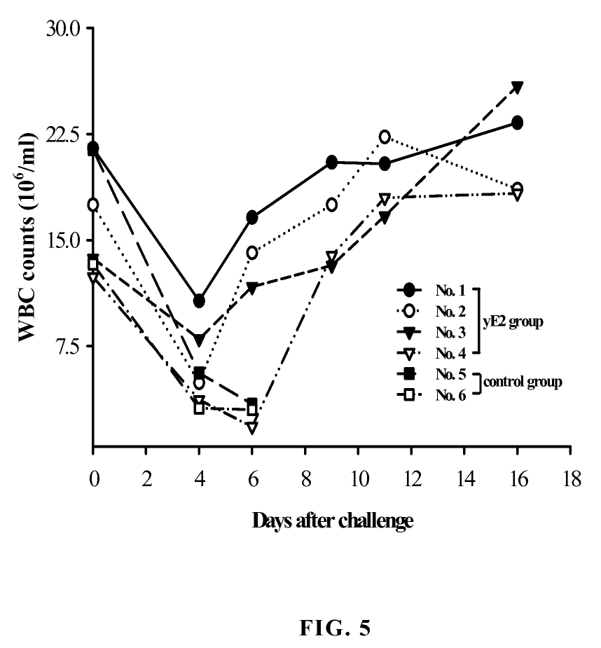

Since CSFV can induce apoptosis in white blood cells, the change in WBC count may be an indicator for CSFV infection. The WBC counts in pigs before and after challenge infection were monitored by semi-automatic blood cell counter (Sysmex F-800). The number of WBC in all viral challenged pigs had lowered to normal value (1.1×10⁷˜2.2×10⁷/ml), while the WBC counts in yE2-vaccinated pigs had a smaller lowering rate than in control pigs, and all recovered to normal value in six to nine days after challenge infection (as shown in FIG. 5). The yE2-vaccinated pigs were sacrificed for anatomical examination at 2 weeks after challenge infection, and showed that no obvious clinical pathology occurred in those pigs. Accordingly, pigs immunized with yE2 subunit vaccine are not only relieved of clinical symptoms of CSFV infection, but also exhibit sufficient immunity against CSFV infection.

Although the neutralizing titer of pig No. 4 in the yE2-vaccinated group had been below 2⁵, and it exhibited a mild febrile response and a slower recovery of WBC count when compared to other pigs in the vaccinated group, the animal had quickly mounted titer of neutralizing antibody up to 2¹⁴ at day 7 after challenge infection. These results indicate that neutralizing antibody elicited by yE2 immunization can neutralize CSFV effectively and protect pigs from viral infection. The titer of neutralizing antibody in all yE2-vaccinated pigs was highly elevated at day 7 after challenge infection. In addition, WBC counts had recovered to normal value at day 9 after challenge infection. All these data indicate that the memory B cells producing neutralizing antibody in pigs vaccinated with yE2 subunit vaccine will be activated and proliferate to produce large amounts of neutralizing antibody after CSFV infection, which further eliminates CSFV in blood and inhibits WBC apoptosis induced by CSFV.

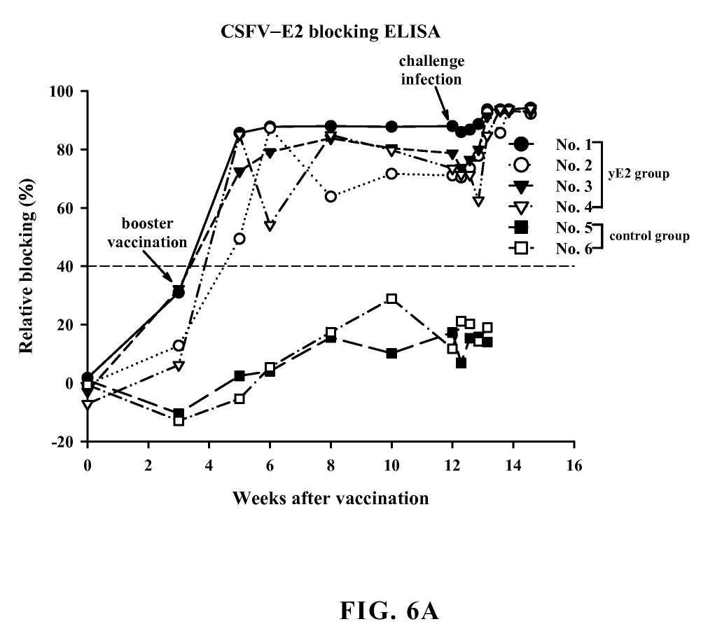

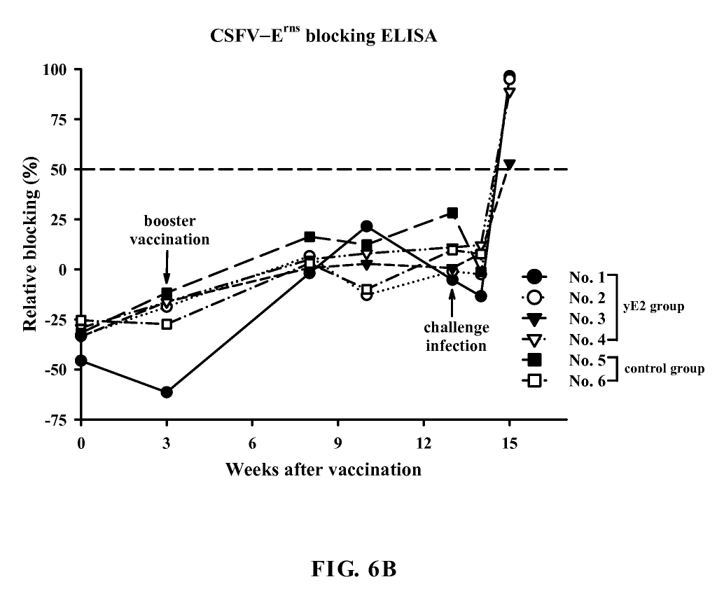

Additionally, the titer of anti-E2 and anti-E^{rns} antibody in serum samples were analysed by using E2 blocking ELISA (CSFV antibody testing kit, Idexx Laboratory) and CSFV marker ELISA (CHEKIT CSFV marker ELISA, Idexx Laboratory), respectively. As shown in FIG. 6, all the yE2-vaccinated pigs seroconverted to CSFV-E2-specific antibody after booster vaccination (FIG. 6A) and revealed clearly E^{rns} antibody negative and seroconverted against E^{rns} by 11 days after challenge infection (FIG. 6B). The result further proves that recombinant yE2 glycoprotein could not only elicit protective effect in pigs, but could also be considered a potential marker vaccine for serological discrimination between vaccinated and infected animals.

The yeast expressed E2 protein (yE2) of the invention possesses glycosylation conformation and correct immunogenicity, and is able to induce production of neutralizing antibody of high titers after vaccination and last for a long period of time, which can provide a protection against lethal dose of CSFV infection without occurrence of clinical side effects. Thus, the yE2 of the invention holds a great potential for development of an efficacious CSF subunit marker vaccine with advantages of exhibiting glycosylation modification, easy manipulation and purification, and low cost.

What is claimed is:

1. A process for producing glycoprotein E2 (yE2) of classical swine fever virus (CSFV) expressed in a recombinant yeast system, which comprises the steps of:
(a) constructing a recombinant expression plasmid (pGAPZαC/E2) by cloning a gene fragment of glycoprotein E2 of classical swine fever virus (CSFV) vector into a yeast expression vector pGAPZαC;
(b) transforming the recombinant expression plasmid obtained in step (a) into Pichia pastoris host cells to yield transformed Pichia pastoris host cells;
(c) cultivating the transformed Pichia pastoris host cells obtained in step (b) to achieve expression and secretion of recombinant yE2 glycoprotein; and
(d) isolating and purifying the recombinant yE2 glycoprotein from a supernatant of a culture medium of step (c).

2. The process of claim 1, wherein the recombinant yE2 glycoprotein is modified by N-glycosylation.

3. The process of claim 1, which is characterized in that the recombinant yE2 glycoprotein produced forms a homodimer and exhibits glycosylation conformation and possesses correct immunogenicity.

4. A subunit vaccine for protecting pigs against CSFV infection, which comprises the recombinant yE2 glycoprotein obtained in the process of claim 1, and a veterinarily acceptable adjuvant.

5. The subunit vaccine of claim 4, which is used as a marker vaccine.

6. The subunit vaccine of claim 5, which does not induce the production of anti-E^{rns} antibody in vaccinated pigs.


SEQUENCE LISTING (4 sequences)

SEQ ID NO: 1 - DNA, 1092 bp, Classical swine fever virus (CSFV)
cggctagcctgcaaggaagactacaggtacgcaatatcgtcaaccgatgagatagggctacttggggccggaggtctcaccaccacctggaaagaatacacccacgatctgcagctgaatgacgggaccgttaaggccacttgcgtggcaggttcctttaaagtcacagcacttaatgtggtcagtaggaggtatttggcatcactgcataagaaggctttacccacttccgtgacattcgaactcctgttcgacgggaccaacccatcaactgaggaaatgggagatgacttcgggttcgggctgtgcccgtttgatacgagacccgttgtcaagggaaagtacaatgcgaccttggtgaacggtagtgctttctatcttgtctgcccaatagggtggacgggtgttatagagtgcacagcagtgagcccaacaaccctgagaacagaagtggtaaagaccttcaggagagacaagccctttccgcacagaatgaattgtgtgaccaccacagtggaaaatgaagacttattctactgtaagttggggggcaactggacatgtgtgaaaggcgaaccagtggtctacacaggggggttagtgaaacaatgtagatggtgtggcttcgacttcaacgagcctgatgggctcccgcactaccccataggtaagtgcattctggcaaacgagacgagttacagagtagtagattcaacggactgcaacagagatggcgttgtaatcagcacagaggggagtcatgagtgcttgattggtaacacgactgtcaaggtgcatgcatcagatgaaagattgggccccatgccatgcagacctaaagagattgtctctagtgcaggacctgcaatgaaaacctcctgtacattcaattacgcaaaaactttgaagaacaggtactatgagcccagggacagctacttccagcaatacatgcttaagggtgagtatcagtactggtttgacctggatgcgactgaccgccactcagattacttcgcagaatttctagaacaaaaactcatctcagaagaggatctgaatagcgccgtcgaccatcatcatcatcatcat

SEQ ID NO: 2 - protein, 364 aa, Classical swine fever virus (CSFV)
RLACKEDYRYAISSTDEIGLLGAGGLTTTWKEYTHDLQLNDGTVKATCVAGSFKVTALNVVSRRYLASLHKKALPTSVTFELLFDGTNPSTEEMGDDFGFGLCPFDTRPVVKGKYNATLVNGSAFYLVCPIGWTGVIECTAVSPTTLRTEVVKTFRRDKPFPHRMNCVTTTVENEDLFYCKLGGNWTCVKGEPVVYTGGLVKQCRWCGFDFNEPDGLPHYPIGKCILANETSYRVVDSTDCNRDGVVISTEGSHECLIGNTTVKVHASDERLGPMPCRPKEIVSSAGPAMKTSCTFNYAKTLKNRYYEPRDSYFQQYMLKGEYQYWFDLDATDRHSDYFAEFLEQKLISEEDLNSAVDHHHHHH

SEQ ID NO: 3 - DNA, 24 bp, Artificial Sequence
  Primer yE2f1
ttatcgattcggctagcctgcaag

SEQ ID NO: 4 - DNA, 23 bp, Artificial Sequence
  Primer yE2dCr
cgctctagaaattctgcgaagta